(19) United States
(12) Patent Application Publication
(10) Pub. No.: US 2005/0060045 A1
(43) Pub. Date: Mar. 17, 2005

(54) Multi-axial prosthetic foot

(76) Inventors: Nolan L. Smith, Sandy, UT (US); Douglas E. Rush, Draper, UT (US); Nathan A. Williams, Heber City, UT (US)

Correspondence Address: Robert B. Leonard;Faegre & Benson LLP, 2200 Wells Fargo Center, 90 South Seventh Street, Minneapolis, MN, 55402-3901, US

(21) Appl. No.: 10/663,514

(22) Filed: Sep. 16, 2003

(51) Int. Cl. A61F 2/66

(52) U.S. Cl. 623/49; 623/55

(57) ABSTRACT

A simple, wide ranging multi-axial foot with energy return and/or storage. An upper member and a spaced apart lower member are joined through a post connected to the lower member and a ball joint connected to the upper member. One buffer is located anterior to the ball joint-post, one buffer is located posterior to the ball joint-post. A heel member and heel buffer provide additional energy storage and return.

### TECHNICAL FIELD

This invention relates to the field of prosthetics and more particularly to joints in prosthetic feet.

### BACKGROUND OF THE INVENTION

Prosthetic devices have evolved through the years from simple blocks of a material placed on a stump of a limb to sophisticated energy storage and return devices which can be formed to look like the missing part. Further, a goal of these improvements is to provide better feedback and therefore more comfort and usefulness to the wearer.

A number of prosthetic feet now include an “ankle.” The prosthetic ankle serves the purpose of allowing the prosthetic foot to move relative to a mounting pylon for attachment to the wearer's stump.

U.S. Pat. No. 817,340 (Rosenkranz) discloses and artificial limb that includes a leg portion 6 and a foot portion 5. A ball 10 and bolt 7 in cooperation with socket 12 are used to connect the leg portion and the foot portion. A cushion block 9 separates the leg portion and foot portion and provides a spring return function when the leg portion and foot portion move relative to the other. While the Rosenkranz prosthesis looked like the part being replaced, it provided only limited energy storage and return capabilities.

U.S. Pat. No. 5,019,109 (Voisin) discloses a prosthetic foot with an ankle plate 12, bottom plate 14 and fore and aft spring members 16 and 18 located between the ankle plate and the bottom plate. The spring members tense or compress upon movement of the ankle plate relative to the lower plate. While the Voisin prosthetic foot provided some multi-axial capability, this capability was limited.

U.S. Pat. No. 5,116,384 (Wilson et al.) discloses a prosthetic foot with a ball joint used as an ankle. The ball joint 24 connects a lower member 12 with an upper lower member 48. An elastic snubber 64 is bonded to the bottom lower member and serves to limit how close the top and bottom lower members can get. However, the Wilson et al. prosthetic device used three ball joints making it difficult and expensive to make.

U.S. Pat. No. 5,112,356 (Harris et al.) discloses a prosthetic foot with a resilient ankle joint. The foot includes an upper member 12A and a lower member 12B. The ankle joint is made from a ball and socket mounted above and connected to the top side of the upper member 12A. The range of motion is limited by an adjusting screw and through use of metal plates structured around the ball in desired locations. While the Harris et al. prosthetic foot allowed for multi-axial movement of the prosthetic foot relative to the pylon, it was complex and required many assembly steps to manufacture.

U.S. Pat. No. 5,181,932 (“Phillips '932) discloses a prosthetic foot with an ankle portion demountably and interchangeably connected thereto. Ankle section 14 connects a foot portion 12 with a pylon 30 and provides a flexible support for the foot potion allowing fore and aft rotational motion of the portion relative to the pylon. While the Phillips '932 prosthetic foot was relatively simple to manufacture, the ankle limited movement of the foot to rotational movement in only one plane.

U.S. Pat. No. 5,571,212 (Cornelius) discloses a prosthetic ankle joint including a foot attachment structure, a pylon attachment structure and a spherical bearing connecting the two attachment structures. The foot attachment structure includes two fins between which the spherical bearing mounts. The pylon attachment structure includes a threaded hole for receiving a threaded rod connected to the spherical bearing. Cushions are placed into holes in the attachment means to limit the movement of the ankle joint. Cornelius did allow for movement of a prosthetic foot in many directions, but it required many steps for manufacturing the attachment structures.

U.S. Pat. No. 5,800,569 (Phillips '569) discloses a prosthetic foot having a upper member, a lower member and an ankle block disposed between and connecting the upper member and the lower member. The ankle block is made of a resilient material that allows movement of the lower member relative to the ankle plate. While the Phillips '569 prosthetic foot allows for motion either fore and aft or laterally, it really does not allow for wide multi-axial motion of the foot relative to the pylon.

U.S. Pat. No. 6,280,479 (Phillips '479) discloses a prosthetic foot having upper and lower members. A block of resilient material 16 is attached between the upper lower member and the lower member. The block is constructed with holes extending through the block into which spring elements may be embedded. However, wide multi-axial function was not achieved.

Soviet Union Patent Application SU 1391643 (Yarolyan et al.) discloses an artificial foot having a lower member and a ball joint. The lower member includes a cylindrical talocrural hinge 2 into which a ball is inserted. The ball is connected to an extension of the wearer's stump. This foot, however does not provide energy storage and return.

PCT patent application WO84/00681 (Itsuro) discloses an artificial foot including a leg portion for attaching to a stump. The leg portion is attached to a foot portion through two hinges, one on either side of the foot. Elastic units 3, 4.1 and 4.2 are used to control motion of the foot relative to the leg portion. The Itsuro prosthetic does not, however, allow movement around more than one axis.

French Patent Publication 2 410 998 (Lebre) discloses a connection between an artificial foot 22 and a leg extension 3. The connection includes a ball joint 30.

While many different multi-axial motion artificial feet have been made, they have been complex and expensive to build, or they lack true multi-axial capability with energy storage and return.

### SUMMARY OF THE INVENTION

The present invention is a wide multi-axial prosthetic foot that is easy to assemble and uses moldable or stock parts and has energy storage and return. An upper member and a lower member can be molded from a lightweight metal such as titanium or can be formed from a fiber reinforced matrix material. A ball joint is added to the lower member, preferably with a bottom opening for the ball joint extending beyond a bottom surface of the upper member. A post is added to the lower member, the post having a diameter such that it may fit within the bottom opening of the ball joint. Buffers, made of a deformable material, are placed between the upper member and the lower member forward and aft of the post to restrict and control the amount of forward and aft rotation of the lower member relative to the upper member.

In an alternative embodiment, buffers can also be placed medially and laterally of the post in order to limit the amount of medial and lateral rotation of the lower member with respect to the upper member.

Further, a heel member and heel buffer may be added to the lower member to increase the energy storage and return function.

### BRIEF DESCRIPTION OF THE DRAWINGS

FIG. 1 is a side view of a prosthetic foot according to the present invention.

FIG. 1A is a top view of the prosthetic foot of FIG. 1.

FIG. 2 is a sectional view taken along line 2-2 of the prosthetic foot of FIG. 1 where the foot includes a single-piece upper member.

FIG. 2A shows the prosthetic foot of FIG. 2 during the toe-off stage of the human walking gait.

FIG. 3 is a sectional view taken along line 2-2 of the prosthetic foot of FIG. 1 where the foot includes a two-piece upper member

FIGS. 4A-4C are side views of the prosthetic foot in stages of the human walking cycle.

FIG. 5A is sectional view taken along line 5-5 of the prosthetic foot of FIG. 1 where the foot includes a single-piece upper member.

FIG. 5B shows the prosthetic foot of FIG. 5A having a medial-lateral response of the foot.

FIG. 5C is sectional view taken along line 5-5 of the prosthetic foot of FIG. 1 where the foot includes a two-piece upper member.

FIG. 6 is a front view of the prosthetic foot of FIG. 1.

FIG. 7 is a side view of an alternate embodiment of the prosthetic foot with addition of a heel member and a buffer.

FIG. 8 is a side view of another alternate embodiment of the prosthetic foot with addition of a heel member and a buffer.

FIG. 9 is a top view of the prosthetic foot of FIG. 8.

FIG. 10 is a sectional view of the prosthetic foot taken along line 10-10 of FIG. 9.

FIG. 11A is an alternate embodiment of the prosthetic foot having perforations and slits in the buffers.

FIG. 11B is another alternate embodiment of the prosthetic foot having hourglass-shaped perforations in the buffers.

FIG. 12A is a side view of another alternate embodiment of the prosthetic foot.

FIG. 12B is a cross-sectional view of the foot of FIG. 11A.

### DETAILED DESCRIPTION OF PREFERRED EMBODIMENT

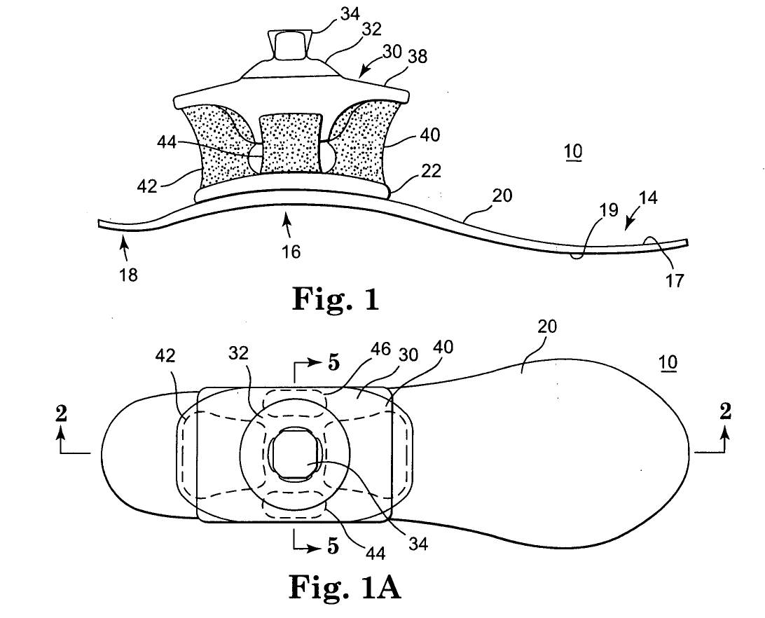

Referring now to FIGS. 1 and 1A, there shown is a side view of the prosthetic foot 10 of the present invention. Lower member 20 provides a base for upper member 30 on which the wearer (not shown) of the prosthetic foot 10 will stand. The lower member 20 includes anterior, medial, and posterior portions 14, 16, and 18. In one embodiment, the lower member 20 can be roughly the size of a human foot and shaped similarly to a human foot. The width of the lower foot member 20 can be variable along its length. In the illustrated embodiment, the width of its anterior portion 14 can be greater than the widths of its medial and posterior portions 16 and 18. In another embodiment, the widths of its anterior and posterior portions 14 and 18 can be greater than the width of its medial portion 16 (not shown). In yet another embodiment, the width of the lower foot member 20 can be constant along its length (not shown).

The lower foot member 20 includes a top surface 17 and a bottom surface 19. In the illustrated embodiment, the lower foot member 20 has a tapered thickness along its length, such that the thickness increases from the posterior portion 18 to the medial portion 16 and decreases from the medial section 16 to the anterior portion 14. The top and bottom surfaces 17 and 19 can be generally curvilinear. The thickness of the lower foot member 14 can be between about 0.08 inches and about 0.40 inches. In another embodiment, the top and bottom surfaces 17 and 19 can be generally planar (not shown).

In an alternative embodiment as shown in FIG. 8, the lower foot member 420 is partially tapered. In particular, the anterior portion 414 and the medial portion 416 can have a tapered thickness along the length of the lower foot member 420, such that the thickness decreases from the medial section 416 to the anterior portion 414, while the posterior portion 418 can have a constant thickness greater than the thickness' of the anterior portion 414 and the medial portion 416.

The lower member 20 can be constructed of any material capable of handling repeated loading and unloading of the wearer's weight. Lightweight materials such as titanium or composite materials such as carbon fiber/epoxy matrix can be used. In order to provide energy return to the wearer, it is preferable that the prosthetic foot 10 be flexible along its length or at least along a portion of its length.

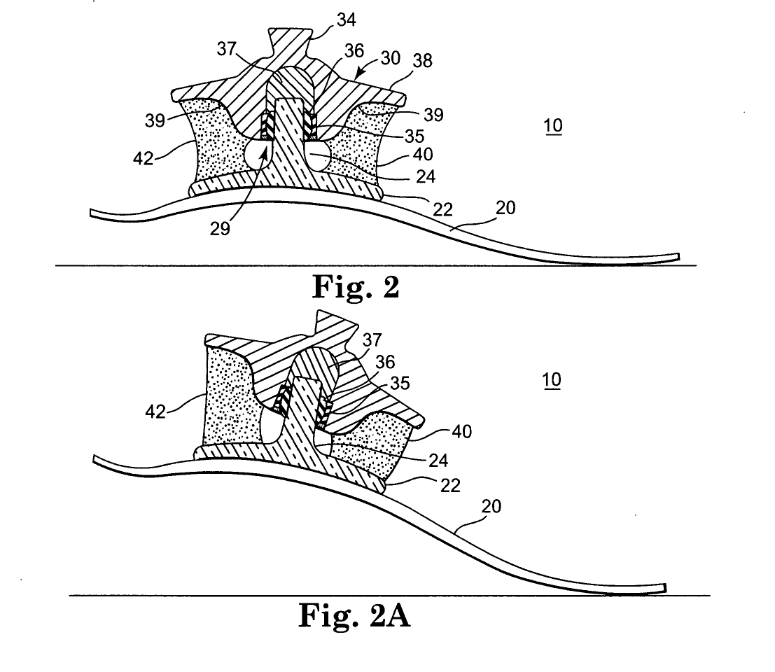

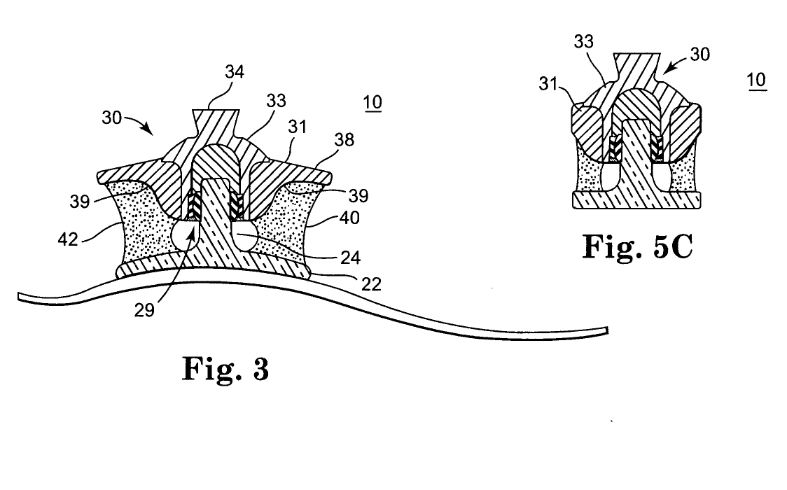

FIG. 2 is a sectional view taken along line 2-2 of the prosthetic foot 10 of FIG. 1A where the prosthetic foot 10 includes a single piece upper member 30. The upper member 30 can be made from titanium, aluminum, or fiber reinforced matrix material. In an alternative embodiment as shown in FIG. 3, the upper member 30 can be a two-piece member including a molded composite part 31 and an insert 33 made of titanium or other lightweight alloy.

Referring to FIGS. 1, 2, and 3, the upper member 30 can be connected to the lower member 20 via post 24. The upper member 30 can serve two primary functions. First, it can provide a stable mounting location for pyramid 34. The pyramid 34 is the attachment point between the prosthetic foot 10 and the stump (not shown) of the wearer. The wearer wears a device, called a pylon (not shown), and the prosthetic foot 10 connects to the pylon via the pyramid 34. Second, the upper member 30 can be sized and shaped to compress or stretch first and second buffers 40 and 42 during the heel-strike and toe-off stages of the walk cycle.

Referring to FIG. 6, there shown is a front view of the prosthetic foot of FIG. 1. The upper member 30 includes a front face 38, which can be sloped.

Referring also to FIGS. 2 and 3, the upper member 30 can be formed with downwardly sloping lower faces 39. The faces 39 serve as mating surfaces for the buffers 40 and 42. By angling the faces 39, a generally linear resistance by the buffers 40 and 42 is noted by the user to flexing in the posterior and anterior directions of the prosthetic foot 10.

The post 24 can be formed as a part of base unit 22. The base unit 22 can be made from stainless steel, titanium or other lightweight alloy. The base unit 22 can be attached to the lower member 20 using one or more of a variety of means including without limitation, adhesives, nut and bolt, rivets, or clamps.

In an alternative embodiment, the post 24 can be formed as part of the lower member 20. In yet another embodiment, the post 24 can be connected to the lower member 20 using one or more of a variety of means including without limitation, adhesives, nut and bolt, rivets, or clamps.

In the embodiments as shown in FIGS. 2 and 3, the first and second buffers 40 and 42 can be placed between lower member 20 and upper member 30, the first buffer 40 being placed on the more anterior part of the prosthetic foot 10 and the second buffer 42 being placed on the more posterior part of the prosthetic foot 10. The buffers 40 and 42 can serve to limit or resist movement of the upper member 30 relative to the lower member 20. Note that no medial or lateral buffers are used in this embodiment. The function served by the medial and lateral buffers is replaced through use of wider first and second buffers. The buffers 40 and 42 each can be formed of an elastomer, such as polyurethane or rubber, or any other deformable material. The buffers 40 and 42 each can also be formed of a spring such as a coil or leaf spring.

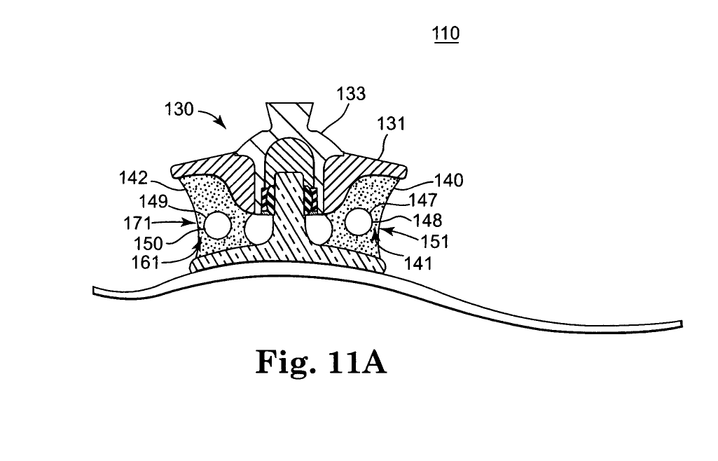

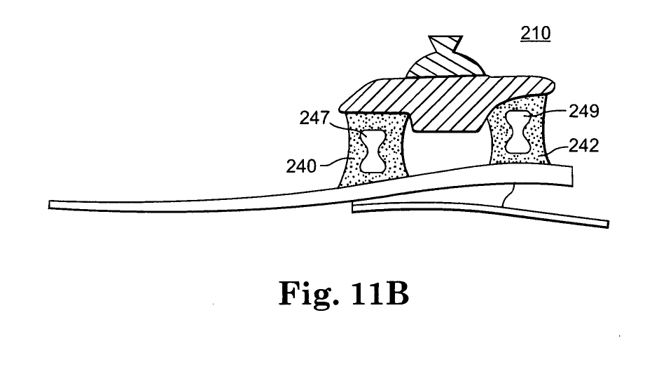

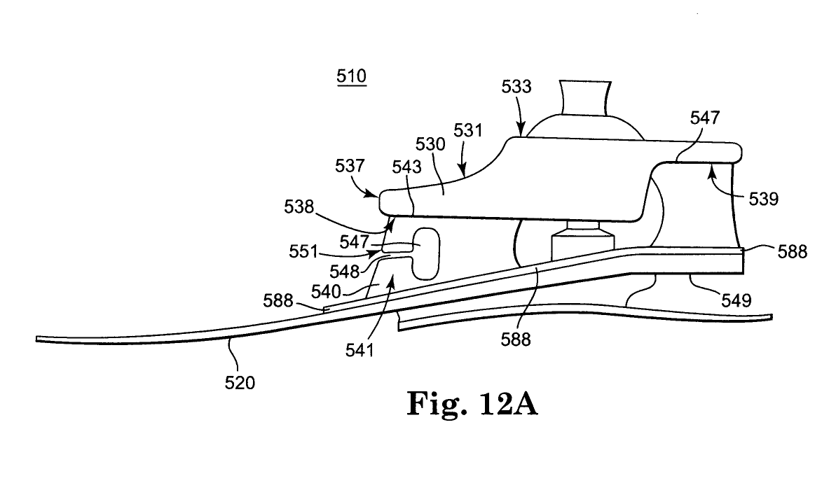

FIG. 11A shows an alternative embodiment of the buffers 40 and 42 of FIG. 3. The buffers 140 and 142 can be constructed to have perforations 147 and 149 laterally formed therethrough, respectively. By placing the perforations 147 and 149 in the buffers 140 and 142, the compressibility of the buffers 140 and 142 is changed. In another embodiment, the buffers 140 and 142 can be constructed to have perforations longitudinally formed therethrough, respectively. In yet another embodiment, the each of the buffers 140 and 142 can be constructed to have two perforations, one laterally formed therethrough and the other longitudinally formed therethrough.

In addition, slits 148 and 150 can be added to modify the tension resistance of the buffers 140 and 142. The slit 148 extends through anterior portion 141 of the first buffer 140 from anterior end 151 to the perforation 147, while slit 150 extends through posterior portion 161 of the second buffer 142 from posterior end 171 to the perforation 149.

Further, each of the perforations 147 and 149 can be filled with a plug (not shown) of a desired durometer, usually a different durometer measurement than that of the buffer material. By inserting such a plug, performance of the prosthetic foot 110 can be “tuned” so that a desired resistance or energy return characteristic may be achieved.

FIG. 11B shows an alternative embodiment of the prosthetic foot 210 in accordance with the present invention. In this embodiment, the buffers 240 and 242 can be constructed to have hourglass-shaped perforations 247 and 249 respectively formed therethrough. The hourglass-shaped perforations 247 and 249 in the buffers 240 and 242 provide resistance during compression of the buffers 240 and 242. The buffers 240 and 242 can be of either lower or higher durometers to provide different resistance to the wearer.

Referring back to FIGS. 2 and 2A, there shown are sectional views of the prosthetic foot 10 of FIGS. 1 and 1A taken along line 2-2. As can be seen from FIG. 2, the post 24 can extend up into a ball joint 29 formed of bearing 36 and socket 35. The bearing 36 is capable of rotation about its center in any direction within socket 35. Gap 37 can be sized so that the end of the post 24 can move a desired amount without contacting the walls of gap 37. When the post 24 is placed in the bearing 36, the upper member 30 can move rotationally about the center of the bearing 36, but cannot move axially along the post 24. In one embodiment, the bearing 36 can extend outside of the upper member 30 and can have a central aperture for receiving the post 24.

In FIG. 2A, the prosthetic foot 10 is shown as if in use and during the toe-off stage of the walking cycle. The first buffer 40 can be compressed from the rest position shown in FIG. 2 due to the rotational movement of the front of the upper member 30 toward the lower member 20. The second buffer 42 can be stretched due to the rotational movement of the back of the upper member 30 away from the lower member 20, thus providing a further limit on the range of motion of the upper member 30 relative to the lower member 20.

In one embodiment, one or both of the buffers 40 and 42 can be connected to both of the upper member 30 and the lower member 20. In an alternative embodiment, one or both of the buffers 40 and 42 can be connected to either the lower member 20 or the upper member 30, but not to both. This would result in one or both of the buffers 40 and 42 only being compressed with no stretching.

In another alternative embodiment, one or both the buffers 40 and 42 can be detachable so as to allow for replacement or adjustment of the buffer durometer. With this modification the prosthetic foot 10 has a rotation control to prevent the upper member 30 from rotating excessively.

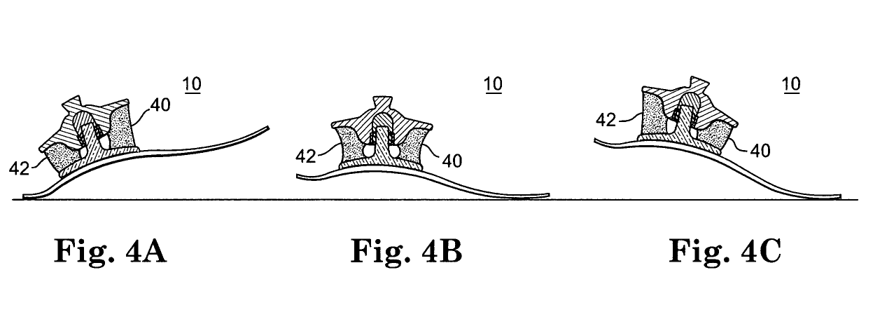

Referring now to FIGS. 4A-4C, there shown are sectional views of a prosthetic foot 10 according to the present invention in all three stages of the human walk cycle, namely, heel-strike, foot-flat, and toe-off. As can be seen in FIG. 4A, during the heel-strike stage, the second buffer 42 can be compressed, while the first buffer 40 can expand. In FIG. 4B, during the foot-flat stage, the first and second buffers 40 and 42 can return to or move to their rest or intermediate positions. In FIG. 4C, during the toe-off stage, the first buffer 40 can be compressed, while the second buffer 42 can expand. The expansion and contraction of the buffers 40 and 42 serve to provide an energy return function to the wearer of the prosthetic foot 10.

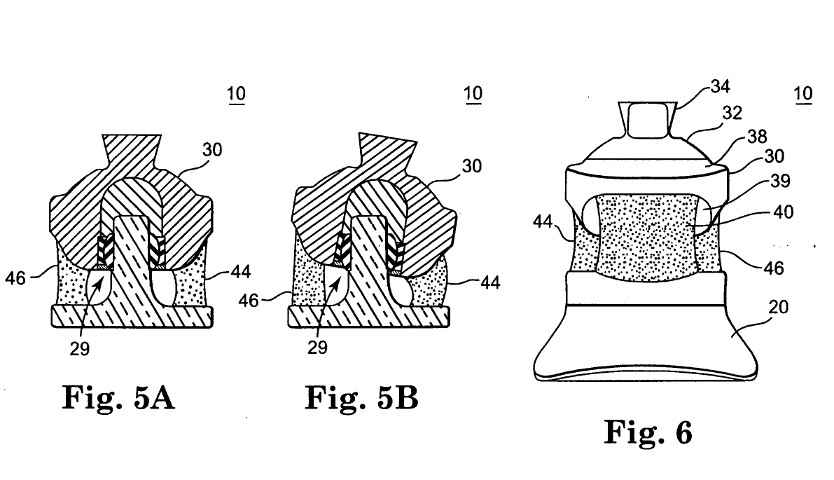

Referring back to FIG. 1A, third and fourth buffers 44 and 46 can be placed on the medial and lateral sides of the prosthetic foot 10. Referring to FIGS. 5A and 5B, there shown are sectional views of the prosthetic foot 10 of FIG. 1A taken along line 5-5. In FIG. 5A, the upper member 30 is in its “normal” position with respect to the lower member (not shown). The upper member 30 has not been rotated medially or laterally with respect to the ball joint 29. In FIG. 5B, the upper member 30 has been rotated medially and the third buffer 44 has been compressed while the fourth buffer 46 has been expanded. The buffer size, shape, and elasticity can be varied to limit the amount of motion. Such changes are design choices depending upon, for example, the desired range of motion and the weight of the wearer.

The third and fourth buffers 44 and 46 each can be formed of an elastomer, such as polyurethane or rubber, or any other deformable material. The buffers 44 and 46 each can also be formed of a spring such as a coil or leaf spring.

In one embodiment, one or both of the third and fourth buffers 44 and 46 can be connected to both of the upper member 30 and the lower member 20. In another embodiment, one or both of the buffers 44 and 46 can be connected to either the lower member 20 or the upper member 30, but not to both. This would result in one or both of the buffers 44 and 46 only being compressed with no stretching. In yet another embodiment, one or both the third and fourth buffers 44 and 46 can be detachable so as to allow for replacement or adjustment of the buffer durometer.

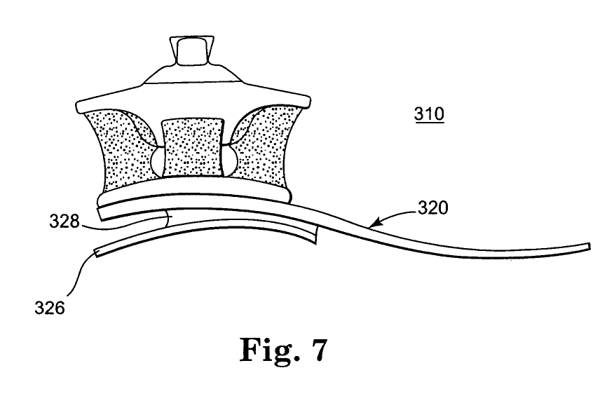

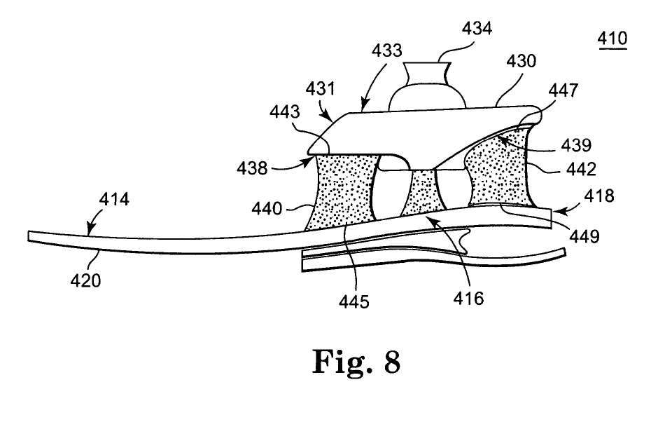

Referring now to FIG. 7, there shown is an alternative embodiment of the prosthetic foot 310 in accordance with the present invention. The prosthetic Foot 310 is similar to the prosthetic foot 10 shown in FIG. 1, with addition of heel member 326 and heel buffer 328. The heel buffer 328 can be positioned between the heel member 326 and the lower member 320. The heel buffer 328 may not fully occupy the space between the lower member 320 and the heel member 326. Further, the heel buffer 328 may not extend anteriorly to the end of the heel member 326. This configuration is designed to return even more energy to the walk cycle of the wearer. The heel member 326 can be of the same material as the lower member 320. The buffer 328 can be of polyurethane, rubber, or other deformable material. In one embodiment, the heel buffer 328 can be bonded to the heel member 326 and the lower member 320.

Referring now to FIGS. 8-10, there shown is yet another embodiment of the prosthetic foot 410 in accordance with the present invention. In this embodiment, only the first and second buffers 440 and 442 are used. The buffers 440 and 442 can be in contact with the lower member 420 and the upper member 430. The post 424 can connect through a smaller base to the lower member 420.

In addition, the upper member 430 can have a different shape. In anterior section 431 of the illustrated embodiment, the upper member 430 can slope from planar top section 433 to anterior bottom section 438. Anterior bottom section 438 can be planar, while posterior bottom section 439 can be curvilinear. The first buffer 440 can have a generally planar top surface 443 and sloped bottom surface 445, while the second buffer 442 can have a curvilinear top surface 447 and a generally planar bottom surface 449.

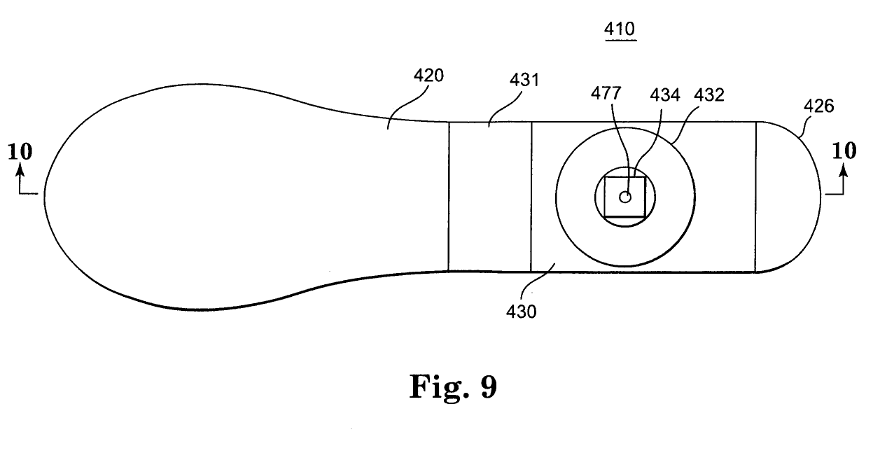

FIG. 9 is a top view of the embodiment shown in FIG. 8. In this view, the lower member 420, the upper member 430, the insert 432, the pyramid 434, and the heel member 426 can be seen. The heel member 426 can extend back beyond the upper member 430 and the lower member 420.

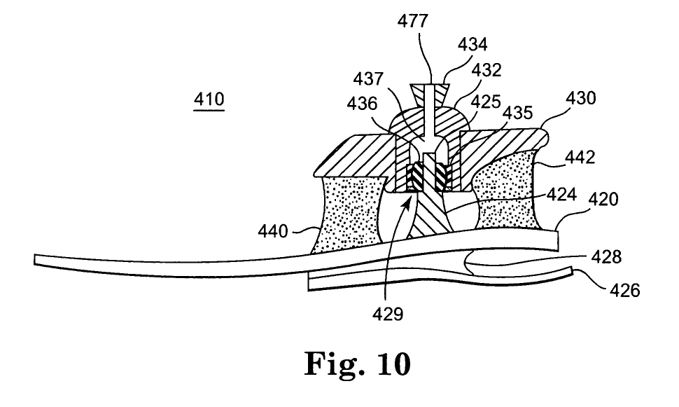

FIG. 10 is a view of the foot of FIGS. 8 and 9 taken along line 10-10. As can be seen, the post 424 includes extension 425 that can extend into ball 436. The ball 436 can be rotationally movable within socket 435. The socket 435 can be held by insert 432. Opening 477 can extend from the pyramid 434 through gap 437 and through the ball 436. Arrangement of the buffers 440 and 442 on the anterior and posterior sides of the ball joint 429 can reduce the complexity of assembly.

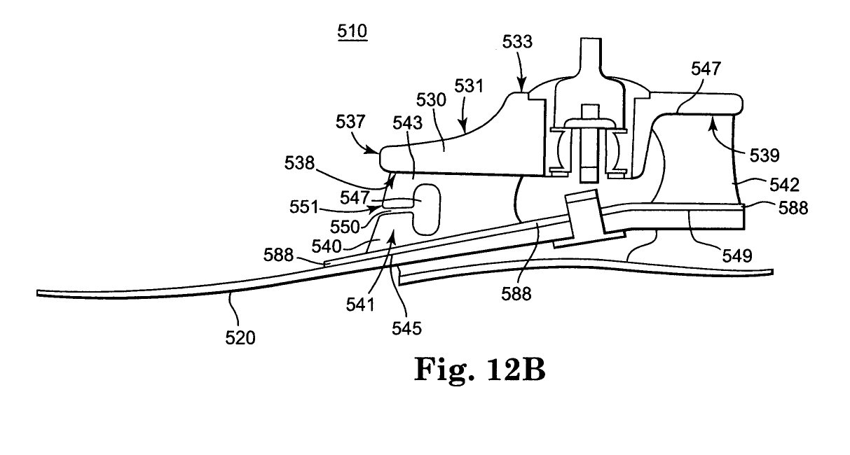

Turning now to FIGS. 12A and 12B, there shown is an alternate embodiment of the prosthetic foot 510 similar to the prosthetic foot 410 of FIG. 8. In this embodiment, perforation 547 can be formed laterally through the first buffer 540. A slit 548 can be formed at the anterior portion 541 of the first buffer 540, such that the anterior portion of the first buffer 540 does not connect the upper member 530 with the lower member 520. The slit 548 extends through anterior portion 541 of the first buffer 540 from anterior end 551 to the perforation 547. During the heel-strike stage of the human walking gait, the slit 548 can allow for further movement of the anterior portion of the upper member 530 away from the lower member 520 than would occur without the slit 548. During the toe-off stage of the human walking gait, there is little or no difference in the compression of the first buffer 540 due to the slit 548.

Similarly to the prosthetic foot 110 shown in FIG. 11A, the perforation 547 of the prosthetic foot 510 can be filled with a plug (not shown) of a desired durometer, usually a different durometer measurement than that of the buffer material. By inserting such a plug, performance of the prosthetic foot 510 can be “tuned” so that a desired resistance or energy return characteristic may be achieved.

In addition, the upper member 530 can have a different shape. In anterior section 531 of the illustrated embodiment, the upper member 530 can slope from planar top section 533 to anterior end section 537. Anterior bottom section 538 and posterior bottom section 539 can be planar. The first buffer 540 can have a generally planar top surface 543 and a sloped bottom surface 545. The bottom surface 549 of the second buffer 542 and a substantial portion of the top surface 547 of the second buffer 542 can be generally planer.

In the illustrated embodiment, the bottom surface 545 of the first buffer 540 and the bottom surface 549 of the second buffer 542 can be coated with a thin layer 588 of the same material as the buffers 40 and 42. This helps dissipate the stresses at the region where the buffers 40 and 42 contact the lower member 20.

The present invention further includes embodiments that vary from the features described above in ways recognized by one of ordinary skill in the art. Without meaning to limit the previous disclosure in any way, use of the words “can,” “can include,” “can be” and the like should be understood to also mean “but need not.

All patents and patent applications disclosed herein, including those disclosed in the background of the invention, are hereby incorporated by reference. Although the present invention has been described with reference to preferred embodiments, workers skilled in the art will recognize that changes may be made in form and detail without departing from the spirit and scope of the invention. In addition, the invention is not to be taken as limited to all of the details thereof as modifications, variations, and different combinations thereof may be made without departing from the spirit or scope of the invention.

1. A prosthetic foot, comprising:
a) a lower member having a top and a bottom;
b) a post extending from the top of the lower member; and
c) An upper member having a ball joint positioned therein, the ball joint having an opening for accepting the post therein.

2. The prosthetic foot of claim 1, further comprising a first buffer positioned between the lower member and the upper member.

3. The prosthetic foot of claim 2, further comprising a second buffer positioned between the lower member and the upper member.

4. The prosthetic foot of claim 3 wherein the lower member and upper member have aligned anterior and posterior regions and the first buffer is located between the anterior regions of the lower member and the upper member and the second buffer is located between the posterior regions of the lower member and the upper member.

5. The prosthetic foot of claim 4, wherein the first and second buffers are formed of deformable material.

6. The prosthetic foot of claim 4, wherein at least one of the first and second buffers includes a perforation extending therethrough.

7. The prosthetic foot of claim 6, wherein the perforation laterally extends through the first buffer.

8. The prosthetic foot of claim 7, wherein the first buffer includes a first slit located at an anterior portion of the first buffer and extending through the anterior portion from an anterior end of the first buffer to the perforation.

9. The prosthetic foot of claim 8, wherein the anterior portion of the first buffer does not connect the upper member with the lower member.

10. The prosthetic foot of claim 6, wherein the perforation longitudinally extends through the first buffer.

11. The prosthetic foot of claim 6, wherein the perforation laterally extends through the second buffer.

12. The prosthetic foot of claim 11, wherein the second buffer includes a second slit located at a posterior portion of the second buffer and extending through the posterior portion from an posterior end of the second buffer to the perforation.

13. The prosthetic foot of claim 6, wherein the perforation longitudinally extends through the second buffer.

14. The prosthetic foot of claim 6 further comprising a plug associated with the perforation.

15. The prosthetic foot of claim 14, wherein the plug is of a material having a durometer different than a durometer of the buffer.

16. The prosthetic foot of claim 6, wherein the perforation is in the shape of an hour glass.

17. The prosthetic foot of claim 4, wherein at least one of the first and second buffers is connected to both of the upper and lower members.

18. The prosthetic foot of claim 4, wherein at least one of the first and second buffers is connected to one of the lower and upper members.

19. The prosthetic foot of claim 4, wherein at least one of the first and second buffers is detachable.

20. The prosthetic foot of claim 4, further comprising third and fourth buffers, wherein both the lower member and the upper member have medial and lateral regions and where the third buffer is located between the medial regions of the lower member and the upper member, and the fourth buffer is located between the lateral regions of the lower member and the upper member.

21. The prosthetic foot of claim 20, wherein the third and fourth buffers are formed of deformable material.

22. The prosthetic foot of claim 20, wherein at least one of the third and fourth buffers is connected to both of the upper and lower members.

23. The prosthetic foot of claim 20, wherein at least one of the third and fourth buffers is connected to one of the lower and upper members.

24. The prosthetic foot of claim 20, wherein at least one of the third and fourth buffers is detachable.

25. The prosthetic foot of claim 1, further comprising a heel member mounted on the bottom of the lower member.

26. The prosthetic foot of claim 25 further comprising a heel buffer positioned between the lower member and the heel member.

27. The prosthetic foot of claim 26, wherein the heel buffer does not fully occupy the space between the lower member and the heel member.

28. The prosthetic foot of claim 26, wherein the heel buffer does not extend anteriorly to the end of the heel member.

29. The prosthetic foot of claim 1, wherein the lower member includes top and bottom surfaces, and the top and bottom surfaces are generally curvilinear.

30. The prosthetic foot of claim 1, wherein the lower member includes anterior, medial, and posterior portions, the lower member has a thickness constant in the posterior portion and decreasing from the medial section to the anterior portion.

31. The prosthetic foot of claim 1, further comprising a base unit attached to the lower member.

32. The prosthetic foot of claim 31, wherein the post and the base unit comprise a unitary structure.

33. The prosthetic foot of claim 31, wherein the post and the base unit comprise separate components.

34. The prosthetic foot of claim 1, further comprising a layer of material coated on a bottom surface of at least one of the first and second buffers wherein the material coated on the bottom surface of the buffer is same as material of the buffer.

35. A prosthetic foot, comprising:
a) a lower member having posterior and anterior regions;
b) an upper member having posterior and anterior regions;
c) a ball joint operably connected to the upper member;
d) a post connected to the lower member, the post connected to the ball joint;
e) a first resilient bumper positioned between the posterior region of the lower member and the posterior region of the upper member; and
f) a second resilient bumper positioned between the anterior region of the lower member and the anterior region of the upper member.

36. The prosthetic foot of claim 35, further comprising a heel member mounted on the bottom of the lower member.

37. The prosthetic foot of claim 36 further comprising a heel buffer positioned between the lower member and the heel member.

38. The prosthetic foot of claim 36, wherein the heel buffer does not fully occupy the space between the lower member and the heel member.

39. The prosthetic foot of claim 37, wherein the heel buffer does not extend anteriorly to the end of the heel member.

40. A prosthetic foot, comprising:
g) a lower member;
h) an upper member;
i) a ball joint operably connected to the upper member;
j) a post connected to the lower member, the post connected to the ball joint;
k) a first resilient bumper positioned between the lower member and the upper member, anteriorly of the ball joint; and
l) a second resilient bumper positioned between the lower member and the upper member, posteriorly of the ball joint.

41. The prosthetic foot of claim 40, further comprising a heel member mounted on the bottom of the lower member.

42. The prosthetic foot of claim 41 further comprising a heel buffer positioned between the lower member and the heel member.

43. The prosthetic foot of claim 42, wherein the heel buffer does not fully occupy the space between the lower member and the heel member.

44. The prosthetic foot of claim 42, wherein the heel buffer does not extend anteriorly to the end of the heel member.
